(19) United States
(12) Patent Application Publication
(10) Pub. No.: US 2026/0279555 A1
(43) Pub. Date: Sep. 17, 2026

(54) SURGEON SWAP CONTROL

(71) Applicant: Digital Surgery Limited, London (GB)

(72) Inventors: Patrick deKlee, London (GB); Davide Volpi, Oxford (GB); Anthony J. Inwood, London (GB)

(21) Appl. No.: 19/473,234

(22) PCT Filed: Apr. 10, 2024
(86) PCT No.: PCT/EP2024/059693

§ 371 Date: Oct. 7, 2025

Related U.S. Application Data
(60) Provisional application No. 63/458,984, filed on Apr. 13, 2023.

(51) Int. Cl. G16H 40/20; G16H 15/00; G16H 20/00; G16H 70/20

(52) U.S. Cl. CPC G16H 40/20; G16H 15/00; G16H 20/00; G16H 70/20

(57) ABSTRACT

An aspect includes a computer-implemented method that associates surgical data with a first surgeon of the surgical team and detects a selection of a surgeon swap control through a user interface during the surgical procedure. An association of the surgical data to a second surgeon of the surgical team is created for subsequently collected surgical data based on the selection. The surgical data captured during the surgical procedure with the associations can be provided to an analytics engine to determine surgical analytics for each of the first surgeon and the second surgeon.

### BACKGROUND

The present disclosure relates in general to computing technology and relates more particularly to computing technology for controlling surgeon association with surgical data generated during a surgical procedure.

Computer-assisted systems, particularly computer-assisted surgery (CAS) systems, can collect and process data digitally during a surgery in an operating room. The data can be stored and/or streamed. In some cases, the data can be used within a system to augment a person's physical sensing, perception, and reaction capabilities. For example, such systems can effectively provide the information corresponding to an expanded field of vision, both temporal and spatial, that enables a person to adjust current and future actions based on the part of an environment not included in his or her physical field of view. The data can be stored and/or transmitted for several purposes such as archival, operational notes, training, post-surgery analysis, and/or patient consultation.

### SUMMARY

According to an aspect, a computer-implemented method provides a surgeon swap control. The method includes associating surgical data with a first surgeon of a surgical team to perform a surgical procedure and detecting a selection of a surgeon swap control through a user interface during the surgical procedure. An association of the surgical data to a second surgeon of the surgical team is created for subsequently collected surgical data based on the selection. The surgical data captured during the surgical procedure with the associations is provided to an analytics engine to determine surgical analytics for each of the first surgeon and the second surgeon.

According to an aspect, a computer program product includes a memory device having computer executable instructions stored thereon, which when executed by one or more processors cause the one or more processors to perform operations including associating surgical data of a surgical procedure with a first surgeon of a surgical team, detecting a selection of a surgeon swap control through a user interface during the surgical procedure, and creating an association of the surgical data for subsequently collected surgical data to a second surgeon of the surgical team based on the selection. The operations further include tracking and recording the surgical data associated with each of the first surgeon and the second surgeon in combination with recording which surgeon is associated with one or more portions of the surgical data.

According to another aspect, a system includes a data reception system configured to capture surgical data of a surgical procedure, a machine learning execution system configured to execute one or more machine-learning models to identify one or more aspects of the surgical procedure as part of the surgical data, and a data correlator. The data correlator is configured to associate a first surgeon selected from a surgeon selection list with the surgical data and the one or more aspects identified of the surgical procedure, and switch to associate a second surgeon selected from the surgeon selection list upon detecting a selection of a surgeon swap control through a user interface during the surgical procedure.

Additional technical features and benefits are realized through the techniques of the present invention. Aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 depicts a computer-assisted surgery (CAS) system according to one or more aspects;

FIG. 2 depicts a surgical procedure system in accordance with one or more aspects.

FIG. 3 depicts a system for storing and analyzing surgical data according to one or more aspects;

FIG. 4 depicts a user interface with a surgeon swap control according to one or more aspects;

FIG. 5 depicts a surgeon selection interface of a surgeon swap control according to one or more aspects;

FIG. 6 depicts a user interface for a data entry and transfer according to one or more aspects;

FIG. 7 depicts a flowchart of a method for surgeon swap control according to one or more aspects;

FIG. 8 depicts a computer system according to one or more aspects; and

FIG. 9 depicts surgeon swap events relative to surgical phases of a surgical procedure according to one or more aspects.

The diagrams depicted herein are illustrative. There can be many variations to the diagrams and/or the operations described herein without departing from the spirit of the invention. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Exemplary aspects of the technical solutions described herein include systems and methods for surgeon swap control during surgical procedures. One or more aspects of the present invention include systems and methods that control associations between a surgeon identifier and surgical data. The surgeon identifier can indicate which surgeon of a plurality of possible surgeons is currently acting in a control capacity during a surgical procedure. In aspects, surgeons acting in a control capacity are physically present at a location where the surgery is performed, e.g., in a same operating room. Tracking an associated surgeon with surgical data generated during a surgical procedure can be useful for later granting access permissions for post-operative access to surgical data and associated medical records (e.g., electronic medical records). For example, in a hospital with multiple surgeons, only those surgeons who were directly involved in performing a surgical procedure may be initially granted access to the surgical data generated during the surgical procedure. The participating surgeons may subsequently decide to grant or revoke access to other medical team members as needed to evaluate the surgical data. Tracking which surgeon was in control during various phases of a surgery can be used to develop surgeon specific metrics and track performance of the surgeon relative to other surgeons. Further, the surgeon specific data can be used as a filter or search term in data extraction. For instance, a surgeon can extract portions of surgical data from a larger data set where the surgeon was the primary actor. This can result in reduced file sizes and reduced network bandwidth consumption by allowing surgeons to filter and extract only portions of surgical data sets in which a particular surgeon was active.

Aspects include a surgeon swap control that allows a currently active surgeon designation to be rapidly changed from one surgeon to another surgeon during a surgical procedure. When a change occurs making a second surgeon become the currently active surgeon, both a first surgeon and the second surgeon can remain associated with the surgical procedure but newly generated surgical data will be associated with the second surgeon until another surgeon swap event happens or the surgical procedure is completed. In some aspects, a surgical team can be tracked together as primary surgeons where at least two surgeons are active as a team and one or more other surgeons are available in a backup role or are performing one or more next tasks.

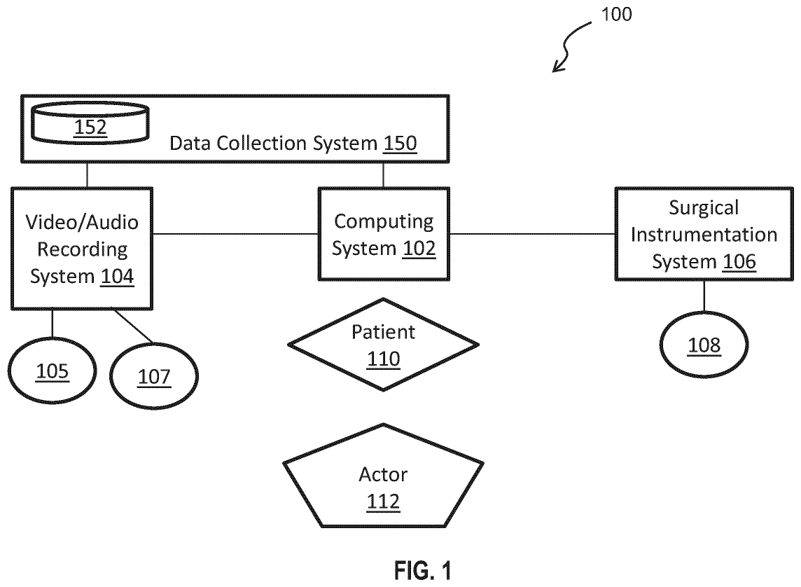

Turning now to FIG. 1, an example computer-assisted surgical (CAS) system 100 is generally shown in accordance with one or more aspects. The CAS system 100 includes at least a computing system 102, a video/audio recording system 104, and a surgical instrumentation system 106. As illustrated in FIG. 1, an actor 112 can be medical personnel that uses the CAS system 100 to perform a surgical procedure on a patient 110. Medical personnel, or health care professionals, can be a surgeon, assistant, nurse, administrator, or any other actor that interacts with the CAS system 100 in a surgical environment. The surgical procedure can be any type of surgery, such as but not limited to open or laparoscopic hernia repair, laparoscopic cholecystectomy, robotic laparoscopic surgery, or any other surgical procedure with or without a robot. In other examples, actor 112 can be a surgeon, anesthesiologist, theatre nurse, technician, an administrator, an engineer, or any other such personnel that interacts with the CAS system 100. For example, actor 112 can record data from the CAS system 100, configure/update one or more attributes of the CAS system 100, review past performance of the CAS system 100, repair the CAS system 100, etc.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g., closure). A “step” refers to the completion of a named surgical objective (e.g., hemostasis). During each step, certain surgical instruments 108 (e.g., forceps) are used to achieve a specific objective by performing one or more surgical actions.

The video/audio recording system 104 shown in FIG. 1 includes one or more cameras 105, such as operating room cameras, endoscopic cameras, etc. The cameras 105 capture video data of the surgical procedure being performed. The video/audio recording system 104 includes one or more video capture devices that can include cameras 105 placed in the surgical room to capture events surrounding (i.e., outside) the patient being operated upon. The video/audio recording system 104 further includes cameras 105 that are passed inside (e.g., endoscopic cameras) the patient 110 to capture endoscopic data. The endoscopic data provides video and images of the surgical procedure.

The video/audio recording system 104 also includes one or more microphones 107, which can be located on a central console, affixed (e.g., via a clip or other means) to medical personnel or objects in the operating room, and/or attached to or integrated into one or more devices in the operating room. Examples of devices in the operating room can include, but are not limited to surgical tools, video recorders, cameras, goggles, personal computers, smart watches, and/or smart phones. The microphones 107 can capture audio data, and can be wired or wireless or a combination of both.

In exemplary aspects, the video data captured by the cameras 105 and the audio data captured by the microphones 107 both include timestamps (or other indicia) that are used to correlate the video data and the audio data. The timestamps can be used to correlate, or synchronize, the sounds captured in the operating room with the images of the medical procedure performed in the operating room. Similarly, timestamps can be used correlate other surgical data collected.

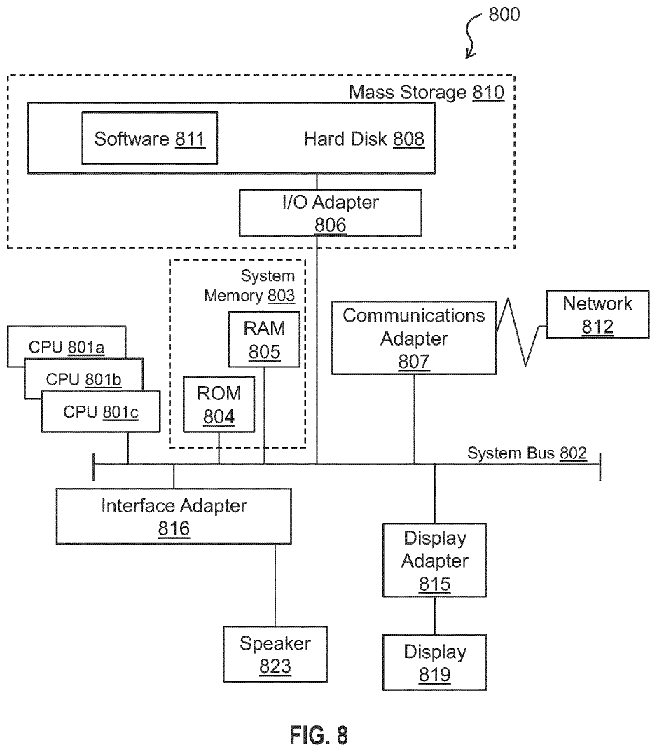

The computing system 102 includes one or more memory devices, one or more processors, and a user interface device, among other components. All or a portion of the computing system 102 shown in FIG. 1 can be implemented for example, by all or a portion of computer system 800 of FIG. 8. Computing system 102 can execute one or more computer-executable instructions. The execution of the instructions facilitates the computing system 102 to perform one or more methods, including those described herein. The computing system 102 can communicate with other computing systems via a wired and/or a wireless network. In one or more examples, the computing system 102 includes one or more trained machine learning models that can detect and/or predict features of/from the surgical procedure that is being performed or has been performed earlier. Features can include structures such as anatomical structures, surgical instruments 108 in the captured video of the surgical procedure. Features can further include events such as phases, actions in the surgical procedure. Features that are detected can further include the actor 112 and/or patient 110. Based on the detection, the computing system 102, in one or more examples, can provide recommendations for subsequent actions to be taken by the actor 112. Alternatively, or in addition, the computing system 102 can provide one or more reports based on the detections. The detections by the machine learning models can be performed in an autonomous or semi-autonomous manner.

The machine learning models can include artificial neural networks, such as deep neural networks, convolutional neural networks, recurrent neural networks, encoders, decoders, or any other type of machine learning model. The machine learning models can be trained in a supervised, unsupervised, or hybrid manner. The machine learning models can be trained to perform detection and/or prediction using one or more types of data acquired by the CAS system 100. For example, the machine learning models can use the video data captured via the video/audio recording system 104. Alternatively, or in addition, the machine learning models use the surgical instrumentation data from the surgical instrumentation system 106. In yet other examples, the machine learning models use a combination of video data and surgical instrumentation data.

After training, the one or more machine-learning models can then be used in real-time to process one or more data streams (e.g., video streams, audio streams, RFID data, etc.). The processing can include predicting and characterizing visualization modifications in images of a video of a surgical procedure based on one or more surgical phases, instruments, and/or other structures within various instantaneous or block time periods. The visualization can be modified to highlight the presence, position, and/or use of one or more structures. Alternatively, or in addition, the structures can be used to identify a stage within a workflow (e.g., as represented via a surgical data structure), predict a future stage within a workflow, etc.

In one or more examples, the machine learning models can detect surgical actions, surgical phases, anatomical structures, surgical instruments, activities, events, and various other features from the data associated with a surgical procedure. The detection can be performed in real-time in some examples. Alternatively, or in addition, the computing system 102 analyzes the surgical data, i.e., the various types of data captured during the surgical procedure, in an offline manner (e.g., post-surgery). In one or more examples, the machine learning models detect surgical phases based on detecting some of the features such as the anatomical structure, surgical instruments, etc.

A data collection system 150 can be employed to store the surgical data, including the video(s) captured during the surgical procedures and the audio data captured during the surgical procedure. The data collection system 150 includes one or more storage devices 152. The data collection system 150 can be a local storage system, a cloud-based storage system, or a combination thereof. Further, the data collection system 150 can use any type of cloud-based storage architecture, for example, public cloud, private cloud, hybrid cloud, etc. In some examples, the data collection system can use a distributed storage, i.e., the storage devices 152 are located at different geographic locations. The storage devices 152 can include any type of electronic data storage media used for recording machine-readable data, such as semiconductor-based, magnetic-based, optical-based storage media, or a combination thereof. For example, the data storage media can include flash-based solid-state drives (SSDs), magnetic-based hard disk drives, magnetic tape, optical discs, etc.

In one or more examples, the data collection system 150 can be part of the video/audio recording system 104, or vice-versa. In some examples, the data collection system 150, the video/audio recording system 104, and the computing system 102, can communicate with each other via a communication network, which can be wired, wireless, or a combination thereof. The communication between the systems can include the transfer of data (e.g., video data, audio data, instrumentation data, etc.), data manipulation commands (e.g., browse, copy, paste, move, delete, create, compress, etc.), data manipulation results, etc. In one or more examples, the computing system 102 can manipulate the data already stored/being stored in the data collection system 150 based on outputs from the one or more machine learning models, e.g., phase detection, structure detection, etc. Alternatively, or in addition, the computing system 102 can manipulate the data already stored/being stored in the data collection system 150 based on information from the surgical instrumentation system 106.

According to aspects, data collection and processing performed by the CAS system 100 can track which surgeon or actor 112 is currently in control of the procedure, such as acting as a primary surgeon. When the role of primary surgeon changes between surgeons, this surgeon swapping event can be captured for subsequent use as surgical data, including video and/or audio data along with instrument data, can be associated with a first surgeon and then switched to a second surgeon during the procedure. Additional surgeon swap events may occur, including switching back to the first surgeon or to one or more other surgeons. The association of particular surgeons with portions of a surgery can be used to determine which surgeon or surgeons should initially be granted read/write/control access to the data captured during the surgical procedure and may be used to filter or selectively extract portions of the data from a larger corpus stored in the one or more storage devices 152 or elsewhere. In some aspects, a surgical team can be tracked together as primary surgeons where at least two surgeons are active as a team and one or more other surgeons are available in a backup role or are performing one or more next tasks.

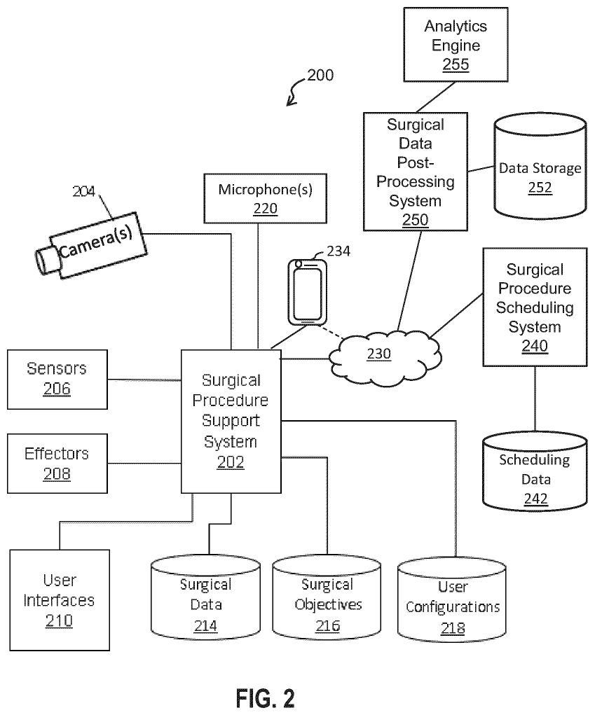

Turning now to FIG. 2, a surgical procedure system 200 is generally shown in accordance with one or more aspects. The example of FIG. 2 depicts a surgical procedure support system 202 that can include or may be coupled to the CAS system 100 of FIG. 1. The surgical procedure support system 202 can acquire image or video data using one or more cameras 204. The surgical procedure support system 202 may also acquire audio data using one or more microphones 220. The surgical procedure support system 202 can further interface with a plurality of sensors 206 and effectors 208. The sensors 206 may be associated with surgical support equipment and/or patient monitoring. The effectors 208 can be robotic components or other equipment controllable through the surgical procedure support system 202. The surgical procedure support system 202 can also interact with one or more user interfaces 210, such as various input and/or output devices. The surgical procedure support system 202 can store, access, and/or update surgical data 214 associated with a training dataset and/or live data as a surgical procedure is being performed on patient 110 of FIG. 1. The surgical procedure support system 202 can store, access, and/or update surgical objectives 216 to assist in training and guidance for one or more surgical procedures. User configurations 218 can track and store user preferences.

The surgical procedure support system 202 can also communicate with other systems through a network 230. For example, the surgical procedure support system 202 can communicate with a surgical procedure scheduling system 240 and/or a surgical data post-processing system 250 through the network 230. Other types of devices, such as a mobile computing device 234 (e.g., a mobile phone, laptop, or tablet computer), can communicate directly with the surgical procedure support system 202 or through the network 230. As one example, user interfaces 210 may be connected to or integrated with the surgical procedure support system 202 by a wired connection while the mobile computing device 234 connects to the surgical procedure support system 202 via a wireless connection. The surgical procedure scheduling system 240 can access and/or modify scheduling data 242 used to track planned surgical procedures. The scheduling data 242 can be used to schedule physical resources and/or human resources to perform planned surgical procedures. For example, the scheduling data 242 can identify two or more surgeons scheduled to be at an operating room of the surgical procedure at a scheduled time of the surgical procedure. The scheduling data 242 can be used to prepopulate scheduling information available for use by the surgical procedure support system 202.

The surgical data post-processing system 250 can receive surgical data and associated data generated by the surgical procedure support system 202 and may be separately stored and secured through data storage 252. For example, surgical data 214 captured during a surgical procedure can be selectively transferred on demand to the data storage 252. Access to specific data or portions of data within the data storage 252 by the surgical data post-processing system 250 may be limited by associated permissions. For instance, only surgeons who actively participated in the surgical procedure may initially have access to the associated data in the data storage 252. The surgical procedure support system 202 can track which surgeons participated with specific portions of the surgical procedure and supply that information within the surgical data 214 or provide the information as metadata associated with the surgical data 214. The surgeons with full access to the data associated with the surgical procedure can subsequently invite other users to view the data through the surgical data post-processing system 250. The surgical data post-processing system 250 may include features such as video viewing, video sharing, data analytics, and selective data extraction. For instance, a surgeon can filter a view to see only the procedures or portions of the procedures in which the surgeon was an active participant. Thus, tracking by surgeon can enhance data security and reduce subsequent processing, network, and storage resource utilization as surgeons can operate on a reduced sized data set where they were active or granted permission by another surgeon who directly participated in a surgical procedure.

In some aspects, analytics engine 255 can be used by or incorporated in the surgical data post-processing system 250 to perform post-operative analytics for each surgeon who participated in the surgery. Post-operative analytics may track times of phases, occurrence of events, and support other types of metrics, such as overall surgical team performance. The resulting analytics can be viewed by the surgeon or member of the surgical team and may be selectively shared with others. In some aspects, the analytics engine 255 can be incorporated with the surgical procedure support system 202 or be accessible by the surgical procedure support system 202 to generate real-time analytics during the surgical procedure. The real-time analytics may be used to trigger an alert where performance is detected to deviate from an expected threshold or range. The ability to track which surgeon or surgeons are associated with particular portions of a surgical procedure can result in more accurate analytics on a surgeon basis.

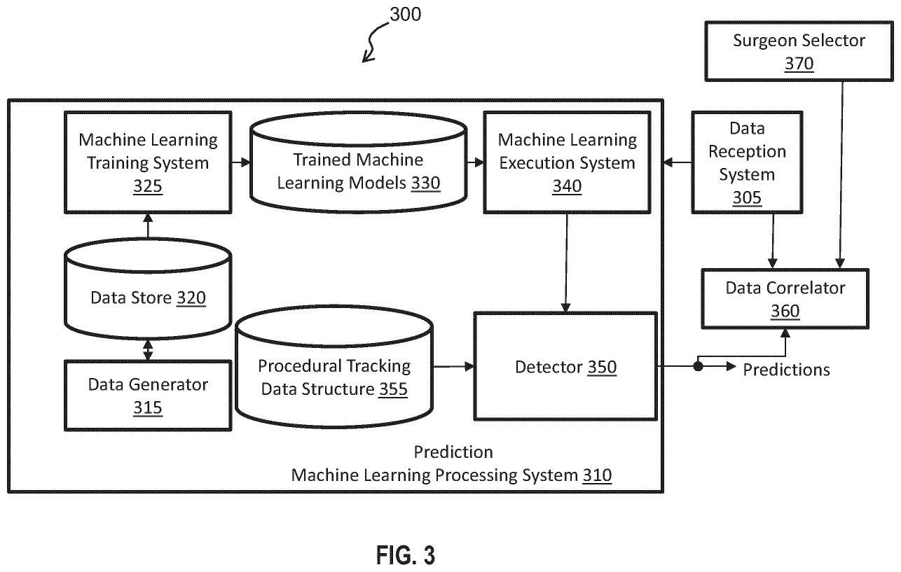

Turning now to FIG. 3, a system 300 for analyzing data that includes video data is generally shown according to one or more aspects. For example, the video data can be captured from video/audio recording system 104 of FIG. 1. The analysis can result in predicting surgical phases and structures (e.g., instruments, anatomical structures, etc.) in the video data using machine learning. System 300 can be the CAS system 100 of FIG. 1, or a part thereof in one or more examples. System 300 uses data streams in the surgical data to identify procedural states according to some aspects.

System 300 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed. Alternatively, or in addition, the data reception system 305 can receive or access surgical data in an offline manner, for example, by accessing data that is stored in the data collection system 150 of FIG. 1.

System 300 further includes a machine learning processing system 310 that processes the surgical data using one or more machine learning models to identify one or more features, such as surgical phase, instrument, anatomical structure, etc., in the surgical data. It will be appreciated that machine learning processing system 310 can include one or more devices (e.g., one or more servers), each of which can be configured to include part or all of one or more of the depicted components of the machine learning processing system 310. In some instances, a part or all of the machine learning processing system 310 is in the cloud and/or remote from an operating room and/or physical location corresponding to a part or all of data reception system 305. It will be appreciated that several components of the machine learning processing system 310 are depicted and described herein. However, the components are just one example structure of the machine learning processing system 310, and that in other examples, the machine learning processing system 310 can be structured using a different combination of the components. Such variations in the combination of the components are encompassed by the technical solutions described herein.

The machine learning processing system 310 includes a machine learning training system 325, which can be a separate device (e.g., server) that stores its output as one or more trained machine learning models 330. The machine learning models 330 are accessible by a machine learning execution system 340. The machine learning execution system 340 can be separate from the machine learning training system 325 in some examples. In other words, in some aspects, devices that “train” the models are separate from devices that “infer,” i.e., perform real-time processing of surgical data using the trained machine learning models 330.

Machine learning processing system 310, in some examples, further includes a data generator 315 to generate simulated surgical data, such as a set of virtual images, or record the video data from the video/audio recording system 104, to train the machine learning models 330. Data generator 315 can access (read/write) a data store 320 to record data, including multiple images and/or multiple videos. The images and/or videos can include images and/or videos collected during one or more procedures (e.g., one or more surgical procedures). For example, the images and/or video may have been collected by a user device worn by the actor 112 of FIG. 1 (e.g., surgeon, surgical nurse, anesthesiologist, etc.) during the surgery, a non-wearable imaging device located within an operating room, or an endoscopic camera inserted inside the patient 110 of FIG. 1. The data store 320 is separate from the data collection system 150 of FIG. 1 in some examples. In other examples, the data store 320 is part of the data collection system 150.

Each of the images and/or videos recorded in the data store 320 for training the machine learning models 330 can be defined as a base image and can be associated with other data that characterizes an associated procedure and/or rendering specifications. For example, the other data can identify a type of procedure, a location of a procedure, one or more people involved in performing the procedure, surgical objectives, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, etc.). Further, the other data can include image-segmentation data that identifies and/or characterizes one or more objects (e.g., tools, anatomical objects, etc.) that are depicted in the image or video. The characterization can indicate the position, orientation, or pose of the object in the image. For example, the characterization can indicate a set of pixels that correspond to the object and/or a state of the object resulting from a past or current user handling. Localization can be performed using a variety of techniques for identifying objects in one or more coordinate systems.

The machine learning training system 325 uses the recorded data in the data store 320, which can include the simulated surgical data (e.g., set of virtual images) and actual surgical data to train the machine learning models 330. The machine learning model 330 can be defined based on a type of model and a set of hyperparameters (e.g., defined based on input from a client device). The machine learning models 330 can be configured based on a set of parameters that can be dynamically defined based on (e.g., continuous or repeated) training (i.e., learning, parameter tuning). Machine learning training system 325 can use one or more optimization algorithms to define the set of parameters to minimize or maximize one or more loss functions. The set of (learned) parameters can be stored as part of a trained machine learning model 330 using a specific data structure for that trained machine learning model 330. The data structure can also include one or more non-learnable variables (e.g., hyperparameters and/or model definitions).

Machine learning execution system 340 can access the data structure(s) of the machine learning models 330 and accordingly configure the machine learning models 330 for inference (i.e., prediction). The machine learning models 330 can include, for example, a fully convolutional network adaptation, an adversarial network model, an encoder, a decoder, or other types of machine learning models. The type of the machine learning models 330 can be indicated in the corresponding data structures. The machine learning model 330 can be configured in accordance with one or more hyperparameters and the set of learned parameters.

The one or more machine learning models 330, during execution, receive, as input, surgical data to be processed and subsequently generate one or more inferences according to the training. For example, the video data captured by the video/audio recording system 104 of FIG. 1 can include data streams (e.g., an array of intensity, depth, and/or RGB values) for a single image or for each of a set of frames (e.g., including multiple images or an image with sequencing data) representing a temporal window of fixed or variable length in a video. The video data that is captured by the video/audio recording system 104 can be received by the data reception system 305, which can include one or more devices located within an operating room where the surgical procedure is being performed. Alternatively, the data reception system 305 can include devices that are located remotely, to which the captured video data is streamed live during the performance of the surgical procedure. Alternatively, or in addition, the data reception system 305 accesses the data in an offline manner from the data collection system 150 or from any other data source (e.g., local or remote storage device).

The data reception system 305 can process the video and/or other data received. The processing can include decoding when a video stream is received in an encoded format such that data for a sequence of images can be extracted and processed. The data reception system 305 can also process other types of data included in the input surgical data. For example, the surgical data can include additional data streams, such as audio data, RFID data, textual data, measurements from one or more surgical instruments/sensors, etc., that can represent stimuli/procedural states from the operating room. The data reception system 305 synchronizes the different inputs from the different devices/sensors before inputting them in the machine learning processing system 310. Synchronization can be achieved by using a common reference clock to generate time stamps alongside each data stream. The clocks can be shared via network protocols or through hardware locking or through any other means. Such time stamps can be associated with any processed data format, such as, but not limited to text or other discrete data created from the audio signal. Additional synchronization can be performed by linking actions, events, or phase segmented that have been automatically processed from the raw signals using machine learning models.

The machine learning models 330, once trained, can analyze the input surgical data, and in one or more aspects, predict and/or characterize structures included in the video data included with the surgical data. The video data can include sequential images and/or encoded video data (e.g., using digital video file/stream formats and/or codecs, such as MP4, MOV, AVI, WEBM, AVCHD, OGG, etc.). The prediction and/or characterization of the structures can include segmenting the video data or predicting the localization of the structures with a probabilistic heatmap. In some instances, the one or more machine learning models include or are associated with a preprocessing or augmentation (e.g., intensity normalization, resizing, cropping, etc.) that is performed prior to segmenting the video data. An output of the one or more machine learning models can include image-segmentation or probabilistic heatmap data that indicates which (if any) of a defined set of structures are predicted within the video data, a location and/or position and/or pose of the structure(s) within the video data, and/or state of the structure(s). The location can be a set of coordinates in an image/frame in the video data. For example, the coordinates can provide a bounding box. The coordinates can provide boundaries that surround the structure(s) being predicted. The trained machine learning models 330, in one or more examples, are trained to perform higher-level predictions and tracking, such as predicting a phase of a surgical procedure and tracking one or more surgical instruments used in the surgical procedure.

While some techniques for predicting a surgical phase (“phase”) in the surgical procedure are described herein, it should be understood that any other technique for phase prediction can be used without affecting the aspects of the technical solutions described herein. In some examples, the machine learning processing system 310 includes a detector 350 that uses the machine learning models to identify a phase within the surgical procedure (“procedure”). Detector 350 uses a particular procedural tracking data structure 355 from a list of procedural tracking data structures. Detector 350 selects the procedural tracking data structure 355 based on the type of surgical procedure that is being performed. In one or more examples, the type of surgical procedure is predetermined or input by actor 112. The procedural tracking data structure 355 identifies a set of potential phases that can correspond to a part of the specific type of procedure.

In some examples, the procedural tracking data structure 355 can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure 355 may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state can indicate a complication (e.g., blood clots, clogged arteries/veins, etc.), pre-condition (e.g., lesions, polyps, etc.). In some examples, the machine learning models 330 are trained to detect an “abnormal condition,” such as hemorrhaging, arrhythmias, blood vessel abnormality, etc.

Each node within the procedural tracking data structure 355 can identify one or more characteristics of the phase corresponding to that node. The characteristics can include visual characteristics. In some instances, the node identifies one or more tools that are typically in use or availed for use (e.g., on a tool tray) during the phase. The node also identifies one or more roles of people who are typically performing a surgical task, a typical type of movement (e.g., of a hand or tool), etc. Thus, detector 350 can use the segmented data generated by machine learning execution system 340 that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, etc.).

The detector 350 outputs the prediction associated with a portion of the video data that is analyzed by the machine learning processing system 310. The prediction is associated with the portion of the video data by identifying a start time and an end time of the portion of the video that is analyzed by the machine learning execution system 340. The prediction that is output can include an identity of a surgical phase, activity, or event as detected by the detector 350 based on the output of the machine learning execution system 340. Further, the prediction, in one or more examples, can include identities of the structures (e.g., instrument, anatomy, etc.) that are identified by the machine learning execution system 340 in the portion of the video that is analyzed. The prediction can also include a confidence score of the prediction. Various types of information in the prediction that can be output may include phases, actions, and/or events associated with a surgical procedure.

A data correlator 360 can be configured to associate an identifier of a currently selected surgeon from surgeon selector 370 with surgical data from the data reception system 305 and one or more aspects identified of the surgical procedure, such as predictions of the detector 350 based on the machine learning execution system 340. For instance, when the surgeon selector 370 identifies a first surgeon as the current surgeon the data correlator 360 synchronizes surgical data from the data reception system 305 with predictions of the detector 350 and an identifier of the first surgeon. When a surgeon swap is detected as a switch of the current surgeon from the first surgeon to a second surgeon selected from a surgeon selection list, subsequent values of surgical data from the data reception system 305 are synchronized with predictions of the detector 350 and associated with the second surgeon. The associations can be captured and transmitted for use by the surgical data post-processing system 250. The associations can be used by the analytics engine 255 to link generated analytics to the surgeon who was performing in a lead capacity and may be determined in real time and/or post-operatively, where the designation of the lead surgeon can change throughout a surgical procedure.

It should be noted that although some of the drawings depict endoscopic videos being analyzed, the technical solutions described herein can be applied to analyze video and image data captured by cameras that are not endoscopic (i.e., cameras external to the patient's body) when performing open surgeries (i.e., not laparoscopic surgeries). For example, the video and image data can be captured by cameras that are mounted on one or more personnel in the operating room, e.g., surgeon. Alternatively, or in addition, the cameras can be mounted on surgical instruments, walls, or other locations in the operating room.

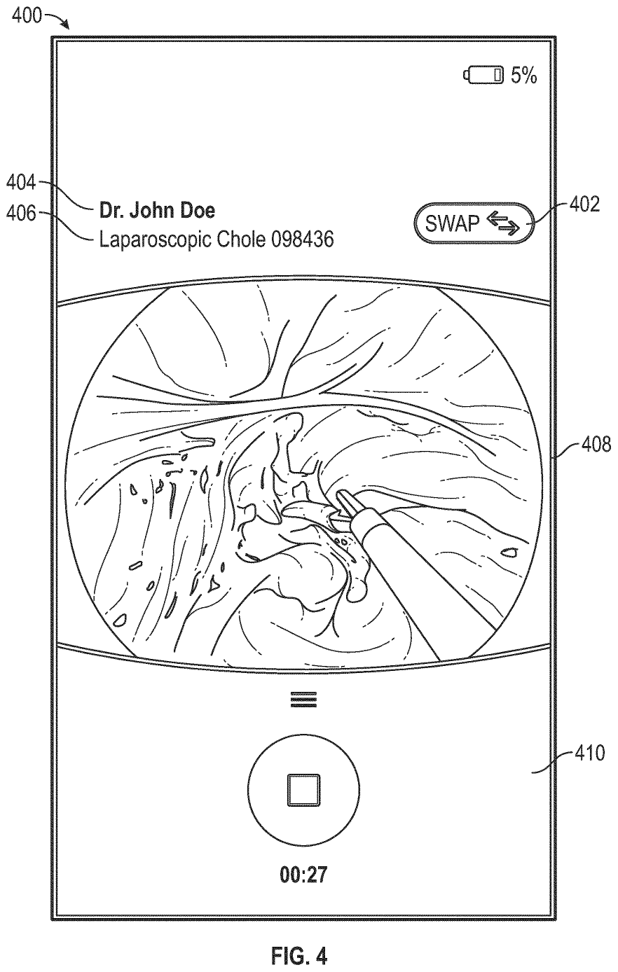

Turning now to FIG. 4, a user interface 400 with a surgeon swap control 402 is depicted according to one or more aspects. The user interface 400 is depicted as a computing device view, such as a mobile computing device 234 paired with a surgical procedure support system 202 of FIG. 2. The user interface 400 can display various types of data, such as a currently active surgeon 404, a surgical procedure 406, a video feed 408 of a camera 204 of FIG. 2, and video controls 410. The surgeon swap control 402 is selectable to change the currently active surgeon 404 designation between a first surgeon and a second surgeon as the second surgeon takes over primary control of the surgery. The video feed 408 can be from a camera within the patient 110, such as an endoscopic camera. The video controls 410 can be selectable to trigger recording of at least a portion of the video feed 408 for a period of time.

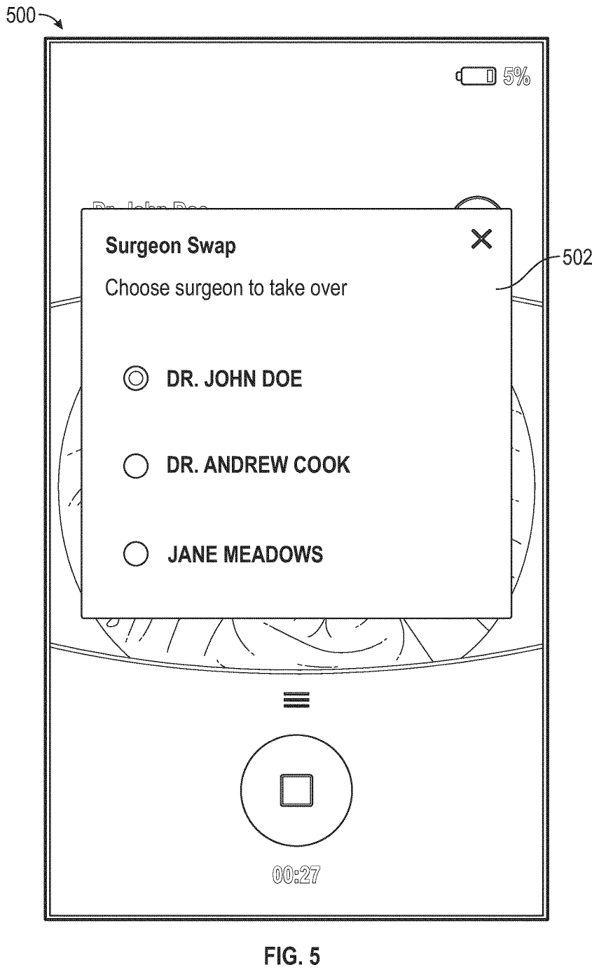

FIG. 5 depicts a surgeon selection interface 500 of a surgeon swap control according to one or more aspects. The surgeon selection interface 500 can be triggered to display a surgeon selection list 502, for instance, upon selecting the surgeon swap control 402 of the user interface 400 of FIG. 4. The surgeon selection list 502 allows a user to rapidly select a different surgeon to take over, such as transitioning from a first surgeon to a second surgeon or any other surgeon included in the surgeon selection list 502. As one example, the surgeon selection list 502 can be a radio button list that is selectable through the surgeon selection interface 500. According to an aspect, the first surgeon, the second surgeon, and one or more additional surgeons can be selectable through the surgeon selection list 502 of the user interface of the surgeon selection interface 500.

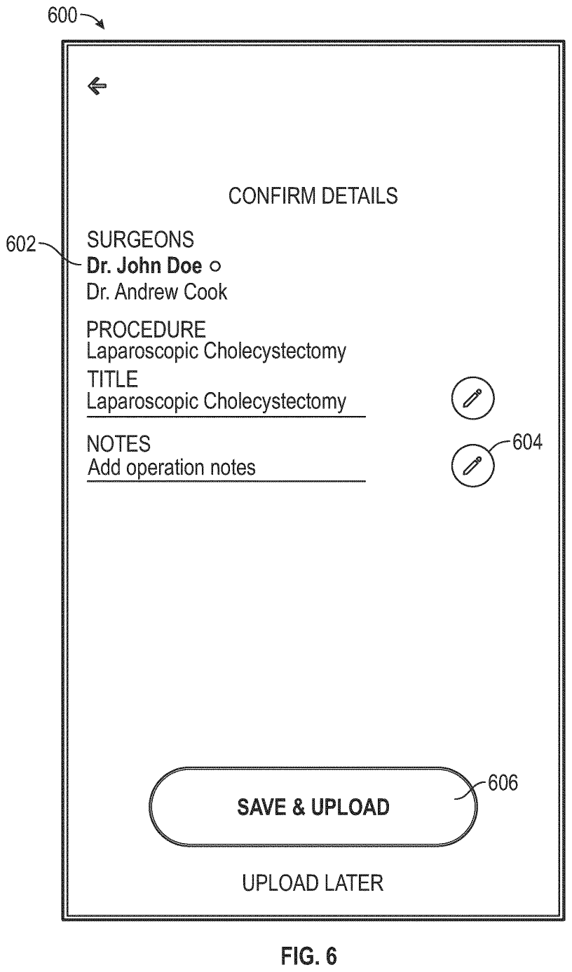

FIG. 6 depicts a user interface 600 for a data entry and transfer according to one or more aspects. The user interface 600 can be triggered for display upon completion of a surgical procedure or selection by a user. The user interface 600 can include a list of participating surgeons 602 who were selected as taking control during a least a portion of the surgical procedure. In some aspects, the list of participating surgeons 602 can indicate which of a plurality of surgeons who participated in the surgical procedure is currently the active surgeon. The user interface 600 can also include editable fields, for instance to change the title of the surgical procedure and/or enter one or more notes 604 about the surgical procedure. Further, the user interface 600 can include an upload selection 606 to trigger an upload of at least a portion of the surgical data to the surgical data post-processing system 250 of FIG. 2. Other options can include a delayed/deferred upload through the user interface 600.

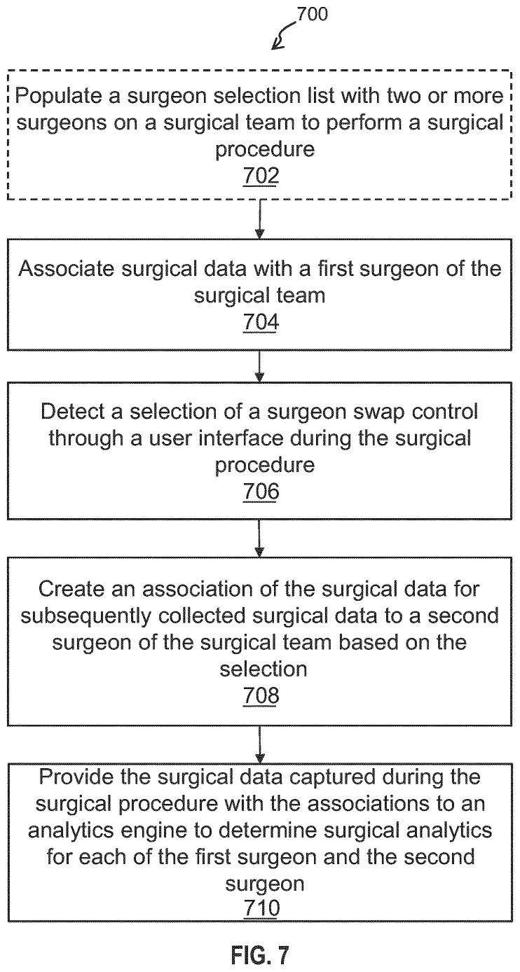

Turning now to FIG. 7, a flowchart of a method 700 for surgeon swap control is generally shown in accordance with one or more aspects. All or a portion of method 700 can be implemented, for example, by all or a portion of CAS system 100 of FIG. 1 and/or computer system 800 of FIG. 8. At block 702, a surgeon selection list 502 can be populated with two or more surgeons on a surgical team to perform a surgical procedure. In other aspects, surgeons can be added during and/or after completion of a surgical procedure. Moreover, selection of a surgeon can be equivalent to selection of a team or sub-team of surgeons where multiple surgeons work together at substantially the same time and/or where at least two surgeons are active as a team and one or more other surgeons are available in a backup role or are performing one or more next tasks. At block 704, surgical data, such as surgical data 214, can be associated with a first surgeon of the surgical team, for instance, as the initial surgeon taking control of the surgical procedure. At block 706, a selection of a surgeon swap control 402 can be detected through a user interface 400 during the surgical procedure to transition a surgeon in control of the surgical procedure from the first surgeon to a second surgeon, where each of the first surgeon and the second surgeon is physically present in at the location of the surgical procedure while in control. At block 708, an association of the surgical data can be created for subsequently collected surgical data to the second surgeon of the surgical team based on the selection. For example, a currently selected surgeon can change from “Dr. John Doe” as the first surgeon to “Dr. Andrew Cook” or “Jane Meadows” as the second surgeon. The surgical data can be augmented by a machine learning system, such as machine learning processing system 310 of FIG. 3.

At block 710, the surgical data captured during the surgical procedure with the associations can be provided to an analytics engine 255 to determine surgical analytics for each of the first surgeon and the second surgeon. The analytics engine 255 can be located local to the surgical procedure support system 202, local to the surgical data post-processing system 250, or may be remotely located, e.g., a cloud-based service. The surgical data with associations can be stored local to the surgical procedure support system 202 or transmitted to another system, such as for storing in data storage 252. Analytics generated by the analytics engine 255 can be determined in real-time while the surgical procedure is still in progress or can be determined post-operatively to further summarize and document the surgical procedure after completion. Tracking which surgeon is directly associated with portions of surgical data can support analytics across multiple procedures. Further, analytics may be used to compare the performance of particular surgical teams when working together. Additionally, tracking associations can be used for filtering of large data sets or customized data extraction for selected surgeons or surgical teams.

According to some aspects, the method 700 can include accessing a surgical procedure scheduling system 240 to identify the two or more surgeons scheduled to be at an operating room of the surgical procedure at a scheduled time of the surgical procedure, for instance, using scheduling data 242. The surgeon selection list 502 can be prepopulated before the surgical procedure begins based on identifying the two or more surgeons. Alternatively, the surgeon selection list 502 can be manually entered or selected from a list that does not receive data from the surgical procedure scheduling system 240. Further, a confirmation user interface, such as user interface 600, can be output that identifies the first surgeon and the second surgeon after selection of the second surgeon. The confirmation user interface, such as user interface 600, can include an operative notes input configured to enter one or more notes 604 about the surgical procedure and an upload selection 606 to trigger an upload of at least a portion of the surgical data to the surgical data post-processing system 250. In some aspects, subsequent access to the surgical data stored by the surgical data post-processing system 250 can be constrained based on an identifier of each of the two or more surgeons who performed the surgical procedure. Further, in some aspects, each of the two or more surgeons who performed the surgery can be provided with permission to invite one or more authorized users to access the surgical data stored by the surgical data post-processing system 250. In some aspects, a revocation control can be provided to revoke access of the one or more authorized users to the surgical data stored by the surgical data post-processing system 250. Further, one or more machine learning models can be executed to identify one or more aspects of the surgical procedure as part of the surgical data. The one or more aspects for each of the two or more surgeons who performed the surgery can be tracked. The one or more aspects can include detecting one or more of: surgical actions, surgical phases, anatomical structures, surgical instruments, activities, and/or events. Aspects may also track remote participants who provided advice during the surgical procedure. Remote participants may also be granted access to post-operative data analysis for at least surgical phases where the remote participants observed or actively participated in providing guidance.

The processing shown in FIG. 7 is not intended to indicate that the operations are to be executed in any particular order or that all of the operations shown in FIG. 7 are to be included in every case. Additionally, the processing shown in FIG. 7 can include any suitable number of additional operations.

Turning now to FIG. 8, a computer system 800 is generally shown in accordance with an aspect. The computer system 800 can be an electronic computer framework comprising and/or employing any number and combination of computing devices and networks utilizing various communication technologies, as described herein. The computer system 800 can be easily scalable, extensible, and modular, with the ability to change to different services or reconfigure some features independently of others. The computer system 800 may be, for example, a server, desktop computer, laptop computer, tablet computer, or smartphone. In some examples, computer system 800 may be a cloud computing node. Computer system 800 may be described in the general context of computer-executable instructions, such as program modules, being executed by a computer system. Generally, program modules may include routines, programs, objects, components, logic, data structures, and so on that perform particular tasks or implement particular abstract data types. Computer system 800 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 8, the computer system 800 has one or more central processing units (CPU(s)) 801a, 801b, 801c, etc. (collectively or generically referred to as processor(s) 801). The processors 801 can be a single-core processor, multi-core processor, computing cluster, or any number of other configurations. The processors 801 can be any type of circuitry capable of executing instructions. The processors 801, also referred to as processing circuits, are coupled via a system bus 802 to a system memory 803 and various other components. The system memory 803 can include one or more memory devices, such as read-only memory (ROM) 804 and a random-access memory (RAM) 805. The ROM 804 is coupled to the system bus 802 and may include a basic input/output system (BIOS), which controls certain basic functions of the computer system 800. The RAM is read-write memory coupled to the system bus 802 for use by the processors 801. The system memory 803 provides temporary memory space for operations of said instructions during operation. The system memory 803 can include random access memory (RAM), read-only memory, flash memory, or any other suitable memory systems.

The computer system 800 comprises an input/output (I/O) adapter 806 and a communications adapter 807 coupled to the system bus 802. The I/O adapter 806 may be a small computer system interface (SCSI) adapter that communicates with a hard disk 808 and/or any other similar component. The I/O adapter 806 and the hard disk 808 are collectively referred to herein as a mass storage 810.

Software 811 for execution on the computer system 800 may be stored in the mass storage 810. The mass storage 810 is an example of a tangible storage medium readable by the processors 801, where the software 811 is stored as instructions for execution by the processors 801 to cause the computer system 800 to operate, such as is described hereinbelow with respect to the various Figures. Examples of computer program product and the execution of such instruction is discussed herein in more detail. The communications adapter 807 interconnects the system bus 802 with a network 812, which may be an outside network, enabling the computer system 800 to communicate with other such systems. In one aspect, a portion of the system memory 803 and the mass storage 810 collectively store an operating system, which may be any appropriate operating system to coordinate the functions of the various components shown in FIG. 8.

Additional input/output devices are shown as connected to the system bus 802 via a display adapter 815 and an interface adapter 816 and. In one aspect, the adapters 806, 807, 815, and 816 may be connected to one or more I/O buses that are connected to the system bus 802 via an intermediate bus bridge (not shown). A display 819 (e.g., a screen or a display monitor) is connected to the system bus 802 by a display adapter 815, which may include a graphics controller to improve the performance of graphics-intensive applications and a video controller. A keyboard, a mouse, a touchscreen, one or more buttons, a speaker, etc., can be interconnected to the system bus 802 via the interface adapter 816, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 8, the computer system 800 includes processing capability in the form of the processors 801, and storage capability including the system memory 803 and the mass storage 810, input means such as the buttons, touchscreen, and output capability including the speaker 823 and the display 819.

In some aspects, the communications adapter 807 can transmit data using any suitable interface or protocol, such as the internet small computer system interface, among others. The network 812 may be a cellular network, a radio network, a wide area network (WAN), a local area network (LAN), or the Internet, among others. An external computing device may connect to the computer system 800 through the network 812. In some examples, an external computing device may be an external web server or a cloud computing node.

It is to be understood that the block diagram of FIG. 8 is not intended to indicate that the computer system 800 is to include all of the components shown in FIG. 8. Rather, the computer system 800 can include any appropriate fewer or additional components not illustrated in FIG. 8 (e.g., additional memory components, embedded controllers, modules, additional network interfaces, etc.). Further, the aspects described herein with respect to computer system 800 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects. Various aspects can be combined to include two or more of the aspects described herein.

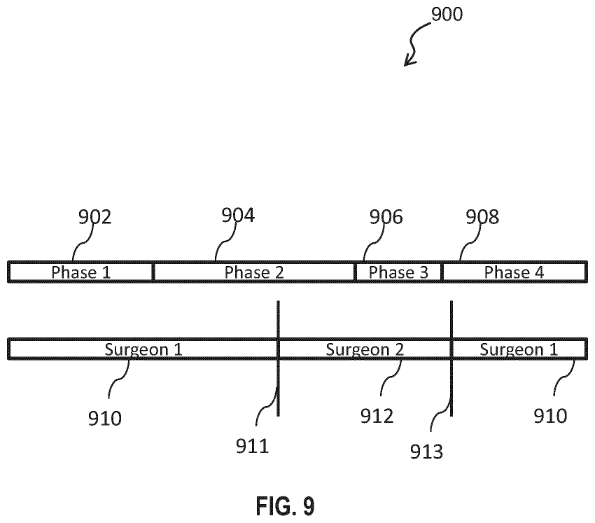

FIG. 9 depicts surgeon swap events relative to surgical phases of a surgical procedure 900 in a time sequence according to one or more aspects. In the example of FIG. 9, during the surgical procedure 900, a plurality of phases, such as phase one 902, phase two 904, phase three 906, and phase four 908 can occur in a time sequence as the surgical procedure 900 progresses. A first surgeon 910 can be initially selected as a surgeon in control for phase one 902 and part of phase two 904 until a surgeon swap event 911 occurs. At that time, a second surgeon 912 can be selected to take over for the first surgeon 910 as the surgeon in control. The second surgeon 912 can be associated with data captured/generated for the second phase 904 in combination with the first surgeon 910 as a shared phase. The second surgeon 912 can be associated with the third phase exclusively. The fourth phase can be initially associated with the second surgeon 912 and then switched at a surgeon swap event 913 to another surgeon, such as switching back to the first surgeon 910 as the surgeon in control. Thus, some associations may involve a complete phase, while others involve a portion of a phase depend when a surgeon swap event occurs relative to surgical phases. At the time of taking control, a surgeon may be physically present at the location of the surgical procedure 900, while a surgeon taking control later in the surgical procedure 900 may not be physically present during earlier or later phases of the surgical procedure 900 (e.g., before or after a surgeon-specific task is performed).

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. The wireless network(s) can include, but is not limited to fifth generation (5G) and sixth generation (6G) protocols and connections. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language such as Smalltalk, C++, high-level languages such as Python, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), graphics processing units (GPUs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A computer-implemented method comprising:
associating surgical data with a first surgeon of a surgical team comprising two or more surgeons to perform a surgical procedure;
detecting a selection of a surgeon swap control through a user interface during the surgical procedure;
creating an association of the surgical data for subsequently collected surgical data to a second surgeon of the surgical team based on the selection; and
providing the surgical data captured during the surgical procedure with the associations to an analytics engine to determine surgical analytics for each of the first surgeon and the second surgeon.

2. The method of claim 1, further comprising:
accessing a surgical procedure scheduling system to identify the two or more surgeons of the surgical team scheduled to be at an operating room of the surgical procedure at a scheduled time of the surgical procedure; and
prepopulating a surgeon selection list before the surgical procedure begins based on identifying the two or more surgeons.

3. The method of claim 1 or claim 2, wherein the analytics engine is configured to generate either or both of real-time analytics and post-operative analytics.

4. The method of any preceding claim, further comprising:
outputting a confirmation user interface that identifies the first surgeon and the second surgeon after selection of the second surgeon.

5. The method of claim 4, wherein the confirmation user interface comprises an operative notes input configured to enter one or more notes about the surgical procedure and an upload selection to trigger an upload of at least a portion of the surgical data to a surgical data post-processing system.

6. The method of claim 5, wherein subsequent access to the surgical data stored by the surgical data post-processing system is constrained based on an identifier of each of the two or more surgeons who performed the surgical procedure.

7. The method of claim 6, further comprising:
providing each of the two or more surgeons who performed the surgical procedure with permission to invite one or more authorized users to access the surgical data stored by the surgical data post-processing system; and
providing a revocation control to revoke access of the one or more authorized users to the surgical data stored by the surgical data post-processing system.

8. The method of any preceding claim, wherein the surgeon swap control tracks at least two surgeons together as primary surgeons who are active as a team and one or more other surgeons are available in a backup role or are performing one or more next tasks.

9. The method of any preceding claim, further comprising:
executing one or more machine learning models to identify one or more aspects of the surgical procedure as part of the surgical data; and
tracking the one or more aspects for each of the two or more surgeons who performed the surgical procedure.

10. The method of claim 9, wherein the one or more aspects comprise detecting one or more of: surgical actions, surgical phases, anatomical structures, surgical instruments, activities, and/or events.

11. A computer program product comprising a memory device having computer executable instructions stored thereon, which when executed by one or more processors cause the one or more processors to perform operations comprising:
associating surgical data of a surgical procedure with a first surgeon of a surgical team;
detecting a selection of a surgeon swap control through a user interface during the surgical procedure;
creating an association of the surgical data for subsequently collected surgical data to a second surgeon of the surgical team based on the selection; and
tracking and recording the surgical data associated with each of the first surgeon and the second surgeon in combination with recording which surgeon is associated with one or more portions of the surgical data.

12. The computer program product of claim 11, wherein the first surgeon, the second surgeon, and one or more additional surgeons are selectable through a surgeon selection list of the user interface.

13. The computer program product of claim 12, wherein the operations further comprise:
accessing a surgical procedure scheduling system to identify three or more surgeons scheduled to be at an operating room of the surgical procedure at a scheduled time of the surgical procedure; and
prepopulating the surgeon selection list before the surgical procedure begins based on identifying the three or more surgeons.

14. The computer program product of any of claims 11 to 13, wherein the operations further comprise:
outputting a confirmation user interface that identifies the first surgeon and the second surgeon, wherein the confirmation user interface comprises an upload selection to trigger an upload of at least a portion of the surgical data to a surgical data post-processing system.

15. The computer program product of claim 14, wherein subsequent access to the surgical data stored by the surgical data post-processing system is constrained based on an identifier of each surgeon who performed the surgical procedure.

16. The computer program product of any of claims 11 to 15, wherein the operations further comprise:
executing one or more machine learning models to identify one or more aspects of the surgical procedure as part of the surgical data; and
tracking the one or more aspects for each surgeon who performed the surgical procedure to filter for events or actions associated with each surgeon during the surgical procedure.

17. A system comprising:
a data reception system configured to capture surgical data of a surgical procedure;
a machine learning execution system configured to execute one or more machine-learning models to identify one or more aspects of the surgical procedure as part of the surgical data; and
a data correlator configured to associate a first surgeon selected from a surgeon selection list with the surgical data and the one or more aspects identified of the surgical procedure, and switch to associate a second surgeon selected from the surgeon selection list upon detecting a selection of a surgeon swap control through a user interface during the surgical procedure.

18. The system of claim 17, wherein the surgeon selection list is populated based on accessing a surgical procedure scheduling system to identify two or more surgeons scheduled to be at an operating room of the surgical procedure at a scheduled time of the surgical procedure.

19. The system of claim 17 or claim 18, wherein the system is configured to output a confirmation user interface that identifies the first surgeon and the second surgeon, wherein the confirmation user interface comprises an upload selection to trigger an upload of at least a portion of the surgical data to a surgical data post-processing system.

20. The system of claim 19, wherein subsequent access to the surgical data stored by the surgical data post-processing system is constrained based on an identifier of each surgeon who performed the surgical procedure.
